(19) United States
(12) Patent Application Publication
(10) Pub. No.: US 2026/0209658 A1
(43) Pub. Date: Jul. 23, 2026

(54) Cannabis Wine: Revolutionizing Fermentation with a Verified Process

(71) Applicant: Leonard R Phillips, Brooklyn, NY (US)

(72) Inventor: Leonard R Phillips, Brooklyn, NY (US)

(21) Appl. No.: 19/281,959

(22) Filed: Jul. 28, 2025

Related U.S. Application Data
(60) Provisional application No. 63/726,289, filed on Jan. 17, 2025.

(51) Int. Cl. C12G 3/02; A61K 9/00; A61K 31/00; A61K 36/185; C12H 1/22

(52) U.S. Cl. CPC C12G 3/02; A61K 9/0095; A61K 31/658; A61K 36/3482; C12H 1/22

(57) ABSTRACT

The invention provides a novel method for producing cannabis wine by fermenting the entire cannabis plant without the use of fruits or post-fermentation infusions. The process involves cleaning and decarboxylating the cannabis plant to activate cannabinoids, combining the prepared material with water and sugars, and fermenting under controlled conditions using yeast strains capable of surviving in cannabinoid-rich environments. After fermentation, the liquid is separated from plant solids, stabilized, and aged to preserve cannabinoids and terpenes. The resulting beverage delivers a naturally balanced profile of alcohol and cannabis-derived compounds, offering a consistent, smooth, and integrated product. This method ensures uniform cannabinoid extraction and natural flavor development while maintaining compliance with safety and quality standards.

### FIELD OF THE INVENTION

The present invention relates generally to the production of alcoholic beverages, and more specifically to a novel method for producing cannabis wine by fermenting the entire cannabis plant without the use of fruits or post-fermentation infusion techniques.

### BACKGROUND OF THE INVENTION

Traditional cannabis-alcohol products often rely on post-fermentation infusion methods, wherein cannabis extracts or oils are blended into an already fermented beverage. These methods frequently produce inconsistent potency, uneven cannabinoid distribution, and nonuniform flavor profiles.

This invention introduces a unique process that ferments the cannabis plant itself, ensuring uniform cannabinoid extraction, natural flavor development, and the creation of a fully integrated cannabis-based alcoholic beverage.

### SUMMARY OF THE INVENTION

The invention provides a method for creating cannabis wine using the entire cannabis plant as the principal ingredient. The process comprises the following steps:

1. Preparation: Cleaning and decarboxylating the cannabis plant to activate cannabinoids.

2. Fermentation: Submerging the prepared cannabis material in water with added sugars and fermenting with selected yeast strains under controlled conditions.

3. Filtration: Removing plant solids post-fermentation while preserving cannabinoids and terpenes.

4. Testing: Ensuring consistent cannabinoid levels and compliance with safety and regulatory standards.

This method enables the direct fermentation of cannabis biomass into a stable, palatable alcoholic beverage while maintaining the natural cannabinoid and terpene profile of the plant.

### DETAILED DESCRIPTION OF THE INVENTION

1. Cannabis Plant Preparation
The process utilizes the entire cannabis plant, including leaves, stems, flowers, roots and seeds.
The harvested cannabis is thoroughly cleaned to remove soil and impurities.
Decarboxylation is performed by heating the plant material at approximately 100° C. to 120° C. for a sufficient period to activate cannabinoids such as THC and CBD, without burning or degrading essential compounds.

2. Fermentation Process

The decarboxylated cannabis material is submerged in water with a suitable sugar source (such as cane sugar, molasses, or glucose).

The mixture is inoculated with selected yeast strains that can thrive in a cannabinoid-rich environment and tolerate moderate alcohol levels.

Fermentation is carried out at controlled temperatures of 18° C. to 22° C., in a dark, warm environment, ensuring optimal yeast activity and cannabinoid integration.

The fermentation continues until the desired alcohol concentration and flavor profile are achieved.

3. Post-fermentation
After fermentation, the liquid is separated from the solid plant material through filtration or pressing, ensuring that active cannabinoids and terpenes remain dissolved in the wine.
The wine may be stabilized to prevent microbial growth and optionally aged to enhance aroma and taste.
The resulting beverage maintains the natural essence of cannabis with a smooth and mellow alcoholic balance.

4. Compliance and Quality Testing
The final product is tested for cannabinoid content, alcohol levels, microbial safety, and regulatory compliance as required by governing authorities.
The method ensures a consistent, high-quality cannabis wine that aligns with both food safety and beverage industry standards.

### STATEMENT REGARDING NO NEW MATTER

The substitute specification includes no new matter.

1. A method for producing cannabis wine comprising: preparing an entire cannabis plant including leaves, stems, flowers, roots and seeds by cleaning and decarboxylating the plant material at a temperature between 100° C. and 120° C. to activate cannabinoids; combining the decarboxylated cannabis plant material with water and a sugar source to form a fermentation mixture; fermenting the mixture at a controlled temperature between 18° C. and 22° C. in a dark and warm environment using a yeast strain capable of fermenting in a cannabinoid-rich medium; separating the liquid phase from the plant solids after fermentation to obtain a fermented cannabis beverage; and
stabilizing, aging, and testing the beverage for cannabinoid content, alcohol concentration, and safety compliance, wherein the resulting product is an alcoholic beverage naturally infused with cannabinoids and terpenes derived directly from the fermented cannabis plant.

2. The method of claim 1, wherein the sugar source comprises at least one of cane sugar, molasses, glucose, or fructose.

3. The method of claim 1, wherein decarboxylation is conducted for a duration sufficient to convert tetrahydrocannabinolic acid (THCA) and cannabidiolic acid (CBDA) to tetrahydrocannabinol (THC) and cannabidiol (CBD) without degradation of active compounds.

4. The method of claim 1, wherein fermentation continues until the beverage achieves an alcohol concentration between 8% and 14% by volume.

5. The method of claim 1, wherein the fermentation mixture is periodically agitated during fermentation to promote even cannabinoid and yeast distribution.

6. The method of claim 1, wherein the yeast strain is selected from Saccharomyces cerevisiae, Brettanomyces bruxellensis, or another strain adapted to tolerate cannabinoids and moderate alcohol levels.

7. The method of claim 1, wherein the step of stabilizing comprises filtration and pasteurization to preserve cannabinoids and terpenes while extending shelf life.

8. The method of claim 1, wherein the aging process enhances the natural cannabis aroma and flavor without addition of external flavoring agents or cannabis extracts.
